(19) United States
(12) Patent Application Publication
(10) Pub. No.: US 2019/0211109 A1
(43) Pub. Date: Jul. 11, 2019

(54) CHRONIC CAR TREATMENT FOR CANCER

(71) Applicant: MaxCyte, Inc., Gaithersburg, MD (US)

(72) Inventors: Madhusudan V. PESHWA, Boyds, MD (US); Linhong LI, North Potomac, MD (US)

(21) Appl. No.: 16/240,382

(22) Filed: Jan. 4, 2019

Related U.S. Application Data
(60) Provisional application No. 62/613,900, filed on Jan. 5, 2018.

(51) Int. Cl. C07K 16/30; A61P 35/00; C07K 14/705; C07K 14/725; A61K 35/17

(52) U.S. Cl. CPC C07K 16/3069; A61P 35/00; A61K 2039/545; C07K 14/7051; A61K 35/17; C07K 14/70578

(57) ABSTRACT

Provided herein are cell populations transiently expressing a chimeric antigen receptor (CAR) and their use in the chronic treatment of hyperproliferative diseases such as cancer.

### CROSS REFERENCE TO RELATED APPLICATIONS

This application claims priority to, and the benefit of, U.S. Provisional Application No. 62/613,900, filed Jan. 5, 2018, the contents of which are incorporated by reference in its entirety for all purposes.

### BACKGROUND

Chimeric antigen receptors (CARs) are used in many clinical applications, including cancer treatment. A CAR is a recombinant receptor composed of an extracellular antigen binding domain and an intracellular T-cell signaling domain. When expressed in T-cells, CARs redirect the T-cells to target the cancer cells that express the targeted antigen in a human leukocyte antigen (HLA)-independent manner. To produce cells expressing a CAR, a nucleic acid encoding the CAR is transfected into an immune cell, and the CAR is then stably expressed in the cell with the antigen-binding region present on the surface. Binding of the antigen-binding region to its target in the subject activates the CAR signaling region in the cytoplasm and causes the immune cell to multiply and elicit an immune response against the cells bearing the antigen, thereby destroying those cells. The use of chimeric antigen receptor (CAR)-modified T cells is an innovative immunotherapeutic approach. CAR cell therapy relies on re-engineering T-cells to express a receptor that allows the cells to recognize targeted cells. Typically, CAR treatment includes collecting T cells from a patient and introducing a chimeric antigen into the collected cells ex vivo, expanding the transfected cells, and then infusing them into a patient.

There are problems with administering stably transfected immune cells expressing a chimeric antigen receptor to patients. First, the CAR-transfected cells may lead to a large, rapid release of cytokines into the blood and cause cytokine release syndrome (CRS) which can lead to fever, nausea, rapid heartbeat, low blood pressure, difficulty breathing, and death. Another potential side effect of CAR therapy is an off-target effect known as B-cell aplasia, where the patient's B cells are killed by the infused CAR cells. To compensate for this side effect, treated patients must receive immunoglobulin therapy for the rest of their lives. Neurotoxicities and brain swelling have also been observed after treatment with stably-transfected CAR-T cells.

### SUMMARY OF THE INVENTION

One method of generating CAR T-cell therapies includes the use of messenger ribonucleic acid (mRNA) to transiently modify mononuclear cells. Using mRNA to re-engineer a patient's mononuclear cells to express a tumor-antigen targeted CAR T-cell can be accomplished in a few hours, allowing on-site preparation and deployment to multiple treatment locations. mRNA CAR mononuclear cells have the safety factor of a limited lifespan, with half-life times similar to antibody therapeutics. Further, these cells lack rapid immune activation and proliferation, thereby limiting the risk for severe cytokine release side effects.

The present disclosure provides a solution to the unwanted and dangerous side-effects observed after stably-transfected CAR treatment by administering to patients cells that transiently express a chimeric antigen receptor. These cells express the CAR for a finite time, in some instances about 7 days. Moreover, as the cells only transiently express the CAR, they may be administered in multiple doses over a longer period of time, thereby providing a chronic treatment to decrease patient symptoms and disease with a diminution, or without, the harmful side effects.

In some aspects, the present disclosure provides methods of treating cancer by chronically administering more than one dose of a population of modified unstimulated mononuclear cells, wherein the unstimulated mononuclear cells are obtained from peripheral blood and transfected with an mRNA encoding a chimeric antigen receptor.

In some embodiments, the dose is repeated daily, weekly, or monthly. In some embodiments, the dose is repeated weekly. In some embodiments, the dose is repeated weekly for at least three weeks. In some embodiments, the dose is 1×10⁷ or 5×10⁷ cells.

In some embodiments, the chimeric antigen receptor comprises an antigen-binding region, a 4-1BB costimulatory signaling region, and a CD3zeta signaling region. In some embodiments, the antigen-binding region is an scFv.

In some embodiments, the antigen-binding region binds to a tumor antigen. In some embodiments, the tumor antigen is an antigen associated with a cancer selected from the group consisting of breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, leukemia, acute lymphocytic leukemia (ALL), acute myelogenous leukemia (AML), chronic lymphocytic leukemia (CLL), chronic myelogenous leukemia (CML), and mantle cell lymphoma (MCL). In some embodiments, the tumor antigen is selected from the group consisting of CD-19, FBP, TAG-72, CEA, CD171, IL-13 receptor, G(D)2, PSMA, mesothelin, Lewis-Y, and CD30.

In some embodiments, the chimeric antigen receptor comprises an anti-mesothelin binding-region. In some embodiments, the anti-mesothelin binding region is an scFv.

In some embodiments, the mononuclear cells are selected from the group consisting of B cells, T cells, Natural Killer cells, or PBMCs.

### BRIEF DESCRIPTION OF THE FIGURES

FIG. 1 demonstrates in vitro expression of a transiently-expressed CAR (MCY-M11).

FIG. 2 demonstrates MCY-M11 inhibits the growth of human mesothelin expressing tumor (ID8) cells in nude mice.

FIG. 3 shows that multiple (weekly) administrations of MCY-M11 result in prolongation of overall survival benefit.

### DETAILED DESCRIPTION

The terms “transient transfection” and “transiently modifying” refer to the introduction of a nucleic acid molecule into a cell using a transfection process that does not y result in the introduced nucleic acid molecule being inserted into the nuclear genome. The introduced nucleic acid molecule is, therefore, lost as the cells undergo mitosis. Any appropriate transfection method may be used. In some embodiments, the transfection method is a physical method. In some embodiments, the transfection method is a chemical method. In some embodiments, the transfection method is a lipofection method. In some embodiments, the transfection method is electroporation. In some embodiments, the transfection method is microfluidics. In some embodiments, the transfection method is a biolistic particle delivery system method (e.g. “gene gun”). In some embodiments, the transfection method is a calcium phosphate transfection method. In some embodiments, the transfection method is selected from the group consisting of, dendrimer assisted transfection, cationic polymer transfection, fugene, nanoparticle assisted transfection, sonoporation, optical transfection, hydrodynamic delivery, impalefection, and particle bombardment. In contrast, “stable transfection” refers to a transfection process in which cells that have integrated the introduced nucleic acid molecule into their genome are selected. In this way, the stably transfected nucleic acid remains in the genome of the cell and its daughter cell after mitosis. The term “transiently expressing” refers to the transient expression of a nucleic acid molecule in a transiently transfected cell.

The use of the term “or” in the disclosure is used to mean “and/or” unless explicitly indicated to refer to alternatives only or the alternatives are mutually exclusive, although the disclosure supports a definition that refers to only alternatives and “and/or.”

The term “about” is used herein to indicate that a value includes the standard deviation of error for the device or method being employed to determine the value.

The words “a” and “an,” when used in conjunction with the word “comprising” in the claims or specification, denotes one or more, unless specifically noted.

The term “chronic administration” as used herein includes the administration of the transiently transfected CAR cells of the disclosure in multiple doses over a period of time (e.g. weekly, monthly, yearly, etc.). In some embodiments, a subsequent dose is not administered until the cells of the previous dose no longer express a CAR. In some embodiments, the transiently transfected CAR cells of the disclosure are administered to a patient until relapse occurs or the patient displays disease progression. In some embodiments, the transiently transfected CAR cells of the disclosure are administered until patient symptoms improve, cancer biomarker expression alters, the cancer size/prevalence is decreased or ameliorated (e.g. a partial response), or the cancer is no longer detectable (e.g. complete response).

The term “unstimulated” (used interchangeably with the term “resting” herein) refers to cells that not been activated, such as by a cytokine or antigen. In some embodiments, the unstimulated cells do not express markers expressed by stimulated cells. In some embodiments, the unstimulated cells do not express PD1, HLA-DR, CD25, CXCR3, and/or CCR4.

Cell Compositions that Transiently Express Chimeric Antigen Receptors

In some aspects, the present disclosure provides compositions comprising or consisting of transiently transfected mononuclear cells made by loading the cells with mRNA instead of DNA. In some embodiments, the compositions are cell populations of transiently transfected mononuclear cells. In some embodiments, the transiently transfected cells are manufactured using the process described in U.S. Pat. No. 9,669,058, which is incorporated herein by reference in its entirety for all purposes.

Loading of cells with mRNA brings several advantages, and overcomes problems associated with DNA transfection, especially in respect to resting cells and cells that will be infused into a patient. First, mRNA results in minimal cell toxicity relative to loading with plasmid DNA. This is especially true for transfection of resting cells such as resting NK and peripheral blood mononuclear cells (PBMC) cells. Also, since mRNA need not enter the cell nucleus to be expressed, resting cells readily express loaded mRNA. Further, since mRNA is not transported to the nucleus, or transcribed or processed, it can begin to be translated essentially immediately following entry into the cell's cytoplasm. This allows for rapid expression of the sequence coded by the mRNA. Moreover, mRNA does not replicate or modify the heritable genetic material of cells. In some embodiments, the mRNA is loaded into the cell via electroporation; various studies on mRNA electroloading have been reported (18-21).

In some embodiments, the present disclosure provides a composition comprising: an transfected mononuclear cell transiently expressing a transgene encoded by a mRNA coding for a chimeric receptor, whereby the chimeric receptor is expressed on the surface of the transfected mononuclear cell; and a pharmaceutically acceptable carrier. In some embodiments, the mononuclear cell is transfected by electroporation. In some aspects of the disclosure, the mononuclear cell is a resting mononuclear cell. In other aspects of the disclosure, the composition is frozen. In some embodiments, the transfected material does not contain a DNA, such as a DNA plasmid, encoding the chimeric receptor or viral vectors or viral-like particles. In certain embodiments, the composition is free or substantially free of non-mononuclear cells. In certain aspects, the composition is about 50% to about 100% free of non-mononuclear cells. In certain aspects, at least about 60%, about 80%, about 90%, about 95%, about 96%, about 97%, about 98%, about 99%, about 99.5%, or about 99.9% of the cells in the composition are mononuclear cells. In some embodiments, the mononuclear cells are PBMCs, PBLs, lymphocytes, B cells, T cells, Natural Killer (NK) cells, or Antigen Presenting Cells (APCs).

The present disclosure provides methods of transfecting mononuclear cells with an mRNA coding for a chimeric antigen receptor, where the entire process from apheresis to cryopreserved cell therapy takes less than one day. In some embodiments, the process for producing modified mononuclear cells includes leukapheresis to obtain cells for manufacturing a modified cell transiently expressing a CAR. In some embodiments, the leukapheresis and transfection of the cells occurs two or more weeks before the start of therapy, and the modified cells are stored by cryopreservation (e.g. stored at −140° C.). In some embodiments, leukapheresis with a yield of at least 5.0×10⁹ cells, and cell processing provides sufficient transfected cells for multiple doses (e.g. at least three weekly doses of 5.0×10⁸ cells).

In some embodiments, this process allows for the transfection of mRNA CAR in up to 20×10⁹ peripheral blood mononuclear cells (PBMCs) for clinical scale manufacture. The cryopreserved cells exhibit expression of a CAR in >95% of cells, which are able to recognize and lyse tumor cells in an antigen-specific manner. Expression of the CAR is detectable over approximately 7-10 days in vitro with a progressive decline of CAR expression that correlates with in vitro cell expansion. These transiently transfected cells target tumors in vivo. For example, in a murine ovarian cancer model, a single IP injection of an anti-mesothelin CAR (MCY-M11) resulted in the dose-dependent inhibition of tumor growth and improved the overall survival of the mice. Further, repeat weekly IP administrations of the optimal dose prolonged disease control and overall survival.

The present disclosure also provides for loading chimeric antigen receptors into PBMCs, and in particular in to antigen presenting cells (APCs), or for loading said chimeric antigen receptors along with other chemical or biological agents that enhance effectiveness of antigen processing, antigen presentation, cell trafficking and localization, and control of immunoregulatory environment in a subject/patient, to facilitate use of freshly isolated (naïve) and modified PBMCs as therapeutic compositions and methods for treatment of cancer and immune diseases.

Mononuclear cells obtained from multiple sources (peripheral blood, bone marrow aspirates, lipo-aspirates, tissue-specific perfusates/isolates) can be effectively loaded with mRNA and chemical and/or biological agents in a controlled manner. In some embodiments, the mononuclear cells are loaded using electrical energy, thereafter referred to as electroloading, to obtain desired level and duration of modulation of molecular pathways. Controlled intervention of molecular pathways provides means for affecting biological activity of cells when administered back to subject/patient, thus enhancing the ability to mitigate potency and efficacy that is otherwise not provided for in the administration of unmodified, freshly isolated cells.

### Natural Killer Cells

In certain embodiments, the present disclosure employs genetically modified natural killer cells in the treatment of hyperproliferative diseases and/or cancer. Natural killer cells (NK cells) are a type of cytotoxic lymphocyte which are activated in response to interferons or macrophage-derived cytokines, and play a major role in the rejection of tumors and cells infected by viruses. NK cells kill cancer cells and virally infected cells by releasing small cytoplasmic granules called perforin and granzyme that cause the target cell to die.

NK cells are characterized by their lack of the T cell receptor (CD3) and their expression of CD56 on their surface. Accordingly, these characteristics may be used to separate NK cells from other cell types. In contrast to cytotoxic T lymphocytes (CTL), NK cells do not require antigen activation and are not MHC restricted.

Cancer cells may evade killing by NK cells because self HLA molecules on the cancer cells can bind to the killer immunoglobulin-like receptors (KIRs) and inhibit the NK cell killing. The present disclosure provides methods and compositions that overcome this inhibition and promotes NK cell killing of cancer cells.

### T Cells

In some embodiments, the present disclosure employs genetically modified T cells, which play a role in cell-mediated immunity, in the treatment of hyperproliferative diseases and/or cancer. One way in which T cells can be distinguished from other lymphocytes, such as B cells and NK cells, is by the presence on their cell surface of the T cell receptor (TCR). Activation of CD8+ T cells and CD4+ T cells occurs through the engagement of both the T cell receptor and CD28 on the T cell by the major histocompatibility complex (MHC) peptide and B7 family members on an antigen presenting cell (APC). Engagement of the T cell receptor for antigen (TCR) in the absence of CD28 costimulation can result in a long-term hyporesponsive state termed clonal anergy (22). Anergic T cells show defective IL-2 production and proliferation upon restimulation via the TCR and CD28, and produce other cytokines at reduced levels. Anergy may represent one mechanism of peripheral tolerance (23), and has been reported to occur in the setting of non-productive anti-tumor immunity in vivo (24).

### Chimeric Antigen Receptors (CARs)

Chimeric antigen receptors generally comprise an extracellular antigen binding domain that recognizes a specific antigen on the target cell surface, and an activation/stimulation domain in the cytoplasm.

The chimeric antigen receptor may include any of several domains, the ectodomain containing a signal peptide or leader sequence and the antigen-binding domain, a spacer region, a transmembrane domain, and an endodomain containing a signaling region. In some embodiments, the CAR includes a leader sequence, an antigen-binding domain, a transmembrane domain, and a signaling domain.

The antigen binding domain may include any domain that will bind to an antigen of interest. In some embodiments, the antigen binding domain contains antibody sequences, variants, or fragments thereof. In some embodiments, the antibody sequences include, but are not limited to, CH1, CH2, or CH3 domains, heavy chains, light chains, scFvs, domain antibodies, a bispecific antibody, CDRs, Fab regions, Fv, Fc regions or fragments thereof. In some embodiments, the antigen-binding domain may be a receptor or ligand sequence or a fragment thereof. In some embodiments, the antigen binding domain binds a tumor antigen or tumor associated antigen.

The antigen binding domain will generally be selected based on the cell being targeted for killing. For example, CD19 is expressed on B-lineage cells, and thus many B-cell cancers. Accordingly, to kill leukemic B cells an anti-CD19 chimeric antigen receptor could be expressed on the surface of a PBMC, such as a NK cell, to enhance interaction between the modified NK cells and the targeted B cells. Thus, in some embodiments, the chimeric antigen receptor is an anti-CD19 chimeric antigen receptor. In some embodiments, the anti-CD19 chimeric antigen receptor is an anti-CD19BBz CAR encoding a single chain antibody conjugated with the 4-1 BB intercellular domain and the CD3ζ domain.

In certain embodiments, the chimeric antigen receptor is an anti-CD20, anti-FBP, anti-TAG-72, anti-CEA, anti-carboxyanhydrase IX, nati-CD171, anti-IL-13 receptor, anti-G(D)2, anti-PSMA, anti-mesothelin, anti-Lewis-Y, or anti-CD30 chimeric antigen receptor. CARs directed to these antigens may be used to treat the diseases associated with the cells that express these antigens. For example, these antigens have been associated with at least the following tumors: CD-19 (leukemia), FBP (ovarian), TAG-72 (colorectal), CEA (colorectal, breast, gastric), carboxyanhydrase IX (renal), CD171 (neuroblastoma), IL-13 receptor (glioblastoma), G(D)2 (neuroblastoma), PSMA (prostate), mesothelin (pancreatic), Lewis-Y (myeloma), or CD30 (cutaneous lymphoma).

The transmembrane domain is fused to the extracellular domain of the CAR. The transmembrane domain may be derived from either a natural or synthetic source. In some embodiments, the transmembrane domain is derived from any membrane-bound or transmembrane protein. In some embodiments, the transmembrane is selected from a group including, but not limited to, the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, or CD154.

In some embodiments, the transmembrane domain may also include a hinge domain. In some embodiments, the hinge domain is a CD8a hinge domain. In other embodiments, the hinge domain is an IgG hinge domain.

The cytoplasmic domain (also called the intracellular signaling domain) of the CAR is responsible for activation of at least one of the normal effector functions of the transfected immune cell. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some embodiments, the intracellular signaling domain is selected from the cytoplasmic sequences of the T cell receptor (TCR) and co-receptors that initiate signal transduction following antigen receptor engagement. In some embodiments, the intracellular signaling domain is selected from a group including, but not limited to, TCR zeta, CD3 zeta, FcR gamma, FcR beta, CD3 gamma, CD3 delta, CD3 epsilon, CD5, CD22, CD79a, CD79b, and CD66d.

In some embodiments, the CARs of the present disclosure include a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include, but are not limited to, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. In certain aspects of the disclosure, the chimeric receptor does not contain an intracellular domain. In certain embodiments, the chimeric receptor does not contain a CD28 intracellular domain.

In some embodiments, the CAR of the present disclosure includes a leader sequence. In some embodiments, the leader sequence is CD8.

In some embodiments, the CAR of the present disclosure includes an anti-mesothelin binding domain, a costimulatory signaling region, and a signaling region. In some embodiments, the CAR of the present disclosure is an mRNA encoding a human anti-Meso ScFv, a CD8a transmembrane region, a 4-1BB costimulatory signaling region, and a CD3 zeta signaling region. In some embodiments, the CAR of the present disclosure is an mRNA encoding a CD8a leader, a human anti-Meso ScFv, a CD8a transmembrane region, a 4-1BB costimulatory signaling region, and a CD3 zeta signaling region.

In some embodiments, the CAR of the disclosure is a human mRNA CAR comprising the peptide domains of scFV-αMESO-H, a transmembrane domain, 4-1BB, and CD3. In some embodiments, the peptide domains are contiguous. In some embodiments, the transfected cell population is non-expanded, autologous peripheral blood mononuclear cells (PBMCs) transfected with mRNA encoding the human CAR of contiguous peptide domains of scFV-αMESO-H, transmembrane domain, 4-1BB, and CD3. This cell population product/therapeutic is also termed MCY-M11. MCY-M11 binds to mesothelin-expressing cells, with subsequent T-cell activation via CD3ζ and costimulatory molecule 4-1BB to activate T-cell dependent antitumor activity.

In some embodiments, chimeric receptor expression in NK, T, PBL, or PBMC cells directly links the NK, T, PBL, or PBMC cells to target cells and consequently allow NK or T cells to kill the target cells. Under this mechanism, the target cell killing can avoid the HLA-type—related NK cell killing inhibition and T cell receptor (TCR)—requirement for T cell-induced target cell killing. In one embodiment of the disclosure, the chimeric receptor is an anti-CD19 chimeric receptor comprising a single chain antibody conjugated with the 4-1 BB intracellular domain and the CD3ζ domain. Chimeric antigen receptor molecules are described in US 2004/0038886, which is incorporated herein by reference in its entirety for all purposes.

### Hyperproliferative Diseases

The compositions of the disclosure may be used in the treatment and prevention of hyperproliferative diseases or hyperproliferative lesions. A hyperproliferative disease is any disease or condition which has, as part of its pathology, an abnormal increase in cell number. Hyperproliferative diseases include, but are not limited to, benign conditions such as benign prostatic hypertrophy and ovarian cysts, as well as premalignant lesions, such as squamous hyperplasia and malignant cancers. Examples of hyperproliferative lesions include, but are not limited to, squamous cell hyperplastic lesions, premalignant epithelial lesions, psoriatic lesions, cutaneous warts, periungual warts, anogenital warts, epidermdysplasia verruciformis, intraepithelial neoplastic lesions, focal epithelial hyperplasia, conjunctival papilloma, conjunctival carcinoma, or squamous carcinoma lesion. A hyperproliferative disease or hyperproliferative lesion can involve cells of any cell type such as keratinocytes, epithelial cells, skin cells, and mucosal cells, and may or may not be associated with an increase in size of individual cells compared to normal cells.

### Cancer

The present disclosure provides methods and compositions for the treatment and prevention of cancer. Cancer is one of the leading causes of death, being responsible for approximately 526,000 deaths in the United States each year. The term “cancer” as used herein is defined as a tissue of uncontrolled growth or proliferation of cells, such as a tumor.

Cancer develops through the accumulation of genetic alterations (25) and gains a growth advantage over normal surrounding cells. The genetic transformation of normal cells to neoplastic cells occurs through a series of progressive steps. Genetic progression models have been studied in some cancers, such as head and neck cancer (26). Treatment and prevention of any type of cancer is contemplated by the present disclosure. The present disclosure also contemplates methods of prevention of cancer in a subject with a history of cancer.

In some embodiments, the compositions and methods disclosed herein may be used to treat cancer or uncontrolled cell growth. In some embodiments, the compositions and methods disclosed herein are used to prevent, inhibit, ameliorate, or decrease metastasis, or uncontrolled cell growth. In some embodiments, the compositions and methods disclosed herein are used to decrease tumor size. In some embodiments, the compositions and methods disclosed herein are used to alter cancer biomarker expression.

In some embodiments, the cancer is a solid cancer. In some embodiments, the cancer is a non-solid cancer. In some embodiments, the disclosure relates to cancers including, but not limited to, acute lymphoblastic leukemia (ALL), acute myeloid leukemia (AML), adrenocortical carcinoma, AIDS-related cancers, anal cancer, appendix cancer, astrocytoma (e.g. childhood cerebellar or cerebral), basal-cell carcinoma, bile duct cancer, bladder cancer, bone tumor (e.g. osteosarcoma, malignant fibrous histiocytoma), brainstem glioma, brain cancer, brain tumors (e.g. cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, Burkitt's lymphoma, carcinoid tumors, central nervous system lymphomas, cerebellar astrocytoma, cervical cancer, chronic lymphocytic leukemia (CLL), chronic myelogenous leukemia (CML), chronic myeloproliferative disorders, colon cancer, cutaneous t-cell lymphoma, desmoplastic small round cell tumor, endometrial cancer, ependymoma, esophageal cancer, Ewing's sarcoma, extracranial germ cell tumor, extragonadal germ cell tumor, extrahepatic bile duct cancer, eye cancer, gallbladder cancer, gastric (stomach) cancer, gastrointestinal stromal tumor (GIST), germ cell tumor (e.g. extracranial, extragonadal, ovarian), gestational trophoblastic tumor, gliomas (e.g. brain stem, cerebral astrocytoma, visual pathway and hypothalamic), gastric carcinoid, head and neck cancer, heart cancer, hepatocellular (liver) cancer, hypopharyngeal cancer, hypothalamic and visual pathway glioma, intraocular melanoma, islet cell carcinoma (endocrine pancreas), kidney cancer (renal cell cancer), laryngeal cancer, leukemias (e.g. acute lymphocytic leukemia, acute myelogenous leukemia, chronic lymphocytic leukemia, chronic myeloid leukemia, hairy cell), lip and oral cavity cancer, liposarcoma, liver cancer, lung cancer (e.g. non-small cell, small cell), lymphoma (e.g. AIDS-related, Burkitt, cutaneous T-cell Hodgkin, non-Hodgkin, primary central nervous system), medulloblastoma, melanoma, Merkel cell carcinoma, mesothelioma, metastatic squamous neck cancer, mouth cancer, multiple endocrine neoplasia syndrome, multiple myeloma, mycosis fungoides, myelodysplastic syndromes, myelodysplastic/myeloproliferative diseases, myelogenous leukemia, myeloid leukemia, myeloid leukemia, myeloproliferative disorders, chronic, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma, ovarian cancer, pancreatic cancer, pancreatic cancer, paranasal sinus and nasal cavity cancer, parathyroid cancer, penile cancer, pharyngeal cancer, pheochromocytoma, pineal astrocytoma and/or germinoma, pineoblastoma and supratentorial primitive neuroectodermal tumors, pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter, retinoblastoma, rhabdomyosarcoma, salivary gland cancer, sarcoma (e.g. Ewing family, Kaposi, soft tissue, uterine), Sézary syndrome, skin cancer (e.g. nonmelanoma, melanoma, merkel cell), small cell lung cancer, small intestine cancer, soft tissue sarcoma, squamous cell carcinoma, squamous neck cancer, stomach cancer, supratentorial primitive neuroectodermal tumor, t-cell lymphoma, testicular cancer, throat cancer, thymoma and thymic carcinoma, thyroid cancer, trophoblastic tumors, ureter and renal pelvis cancers, urethral cancer, uterine cancer, uterine sarcoma, vaginal cancer, visual pathway and hypothalamic glioma, vulvar cancer, Waldenström macroglobulinemia, and Wilms tumor. In some embodiments, the cancer cell expresses mesothelin. In some preferred embodiments, the cancer is selected from the group consisting of ovarian cancer, epithelial ovarian cancer, primary peritoneal carcinoma, fallopian tube carcinoma, peritoneal mesothelioma, pleural mesothelioma, non-small cell lung cancer (squamous or non-squamous), triple negative breast cancer, colorectal cancer, biliary tract cancer, gastric cancer, gastroesophageal cancer, pancreatic cancer, and thymic carcinoma.

In some embodiments, the cancer is refractory or resistant to treatment. In some embodiments, the cancer is in relapse or has progressed. In some embodiments, the cancer is in remission. In some embodiments, the cancer has demonstrated a partial response.

### Mesothelin and Cancer Immunotherapy

Ovarian Cancer

Ovarian cancer typically includes tumors of the ovary, primary peritoneum, or fallopian tube. Collectively this grouping of tumors commonly described as ovarian cancer are among the five most common cancers in women and ranks as fifth as the cause of cancer death in the United States. According to the NIH SEER data of 2017, it is estimated that 22,440 women will be diagnosed with and 14,080 women will die of cancer of the ovary in the US.

Approximately 90% of these women have high grade serous adenocarcinoma of the ovary, primary peritoneum, or fallopian tube. Expression of mesothelin occurs in greater than 80% of epithelial ovarian cancers [2]. Although over 70% of women with advanced disease respond to optimal debulking surgery followed by platinum-taxane based chemotherapy, duration of response is typically less than 2 years and relapse is common. Subsequent responses to salvage therapy regimens tend to be brief (less than six months) due to the tumors' progressive resistance to chemotherapy. Relapsed platinum-resistant ovarian cancers represent a significant challenge. Objective response rates to second-line therapies such as doxorubicin, topotecan and gemcitabine are in the range of 20% and median overall survival is less than 1 year [3-5].

Patients with platinum-resistant ovarian cancer oftentimes have progressive disease with extensive peritoneal disease. Current standard chemotherapy options are rarely effective and have short-term benefit at best. Patients with advanced disease are appropriate candidates for clinical trials for investigational agents.

Malignant Peritoneal Mesothelioma

Malignant peritoneal mesothelioma (MPM) is a rare type of mesothelioma that arises from the serous surfaces of either the visceral or parietal peritoneum and represents approximately 30% of all mesotheliomas [6]. There are three basic histology types including epitheliod (the most frequent), sarcomatoid, or biphasic. Sarcomatoid mesothelioma is extremely rare. Expression of mesothelin occurs in nearly 100% of patients with epitheliod mesothelioma. Patients with biphasic type mesothelioma have variable levels of mesothelin expression depending on the percentage of the epithelial component. Sarcomatoid mesothelioma demonstrates low expression of mesothelin.

In the United States, the overall prevalence is approximately 1 to 2 cases per million with an estimated incidence of 200 to 400 new cases annually. While rare, the incidence has increased over the past two decades, associated with the principal risk factor for increased exposure to asbestos. It can occur in any age group although the 50 to 69 year age group has the highest prevalence, with more common presentation among men, thought secondary to higher male occupational exposure to asbestos.

Patients with MPM have an extremely poor prognosis with a median survival of 6 to 12 months. While surgical resection is the optimal therapy for MPM, most patients present with advanced disease that is not resectable. Patients with unresectable disease are offered first line chemotherapy for palliative intent with pemetrexed in combination with a platinum-based agent such as cisplatin or carboplatin. Patients with progressive disease demonstrate limited and short-term responses with subsequent chemotherapies and are appropriate for investigational therapies through clinical trials.

Mesothelin and Cancer Immunotherapy

The full-length mesothelin gene encodes a 71-kDa precursor protein that is processed to a 31-kDa soluble shed fragment called megakaryocyte potentiating factor (MPF) and a 40-kDa membrane-bound protein termed mesothelin (MESO). MESO is highly expressed in many human cancers, including high grade serous adenocarcinoma of the ovary (75%), pancreatic adenocarcinoma (85%), triple negative breast cancer (66%), and epitheliod mesothelioma (95%) [7].

While the function of MESO on normal cells is non-essential, the expression of MESO on cancer cells may contribute to the pathology of cancer, with higher expression associated with poorer prognosis, increased metastatic spread, and activation of cell growth pathways [7]. MESO provides a significant opportunity for therapeutic targeting for patients with MESOexpressing malignancy, while having a low risk for toxicity of normal cells expressing MESO.

This opportunity for significant therapeutic index is due to the non-essential function of MESOexpressing mesothelial cells throughout the body.

Meso-targeted CAR T-cells using mRNA have demonstrated significant promise in preclinical studies and clinical studies [10-12] by intratumoral, intraperitoneal (IP) and intravenous (IV) of routes of administration. Importantly, Meso-targeted mRNA CAR T-cells demonstrated antitumor feasibility, tolerability and efficacy in preclinical studies with repetitive dosing. A Phase 1 clinical study of CAR T-cells engineered to target MESO using mRNA modification at the University of Pennsylvania demonstrated feasible and safe treatment of 6 patients with pancreatic cancer [10, 11]. This study demonstrated that 53 of 54 planned CAR T-cell infusions were administered by IV therapy, with excellent tolerability, and without evidence of cytokine release syndrome (CRS) that has predominantly limited the viral vector approach to CAR T-cell therapy in B-cell malignancies. Additionally, there was no evidence of on-target/off-tumor mesothelin-related toxicities in normal tissues, without reported toxicities of the pleura, pericardium, or peritoneum. There was also evidence for promising clinical activity with radiological signs of anti-tumor activity. Correlative pharmacodynamic studies also were supportive, demonstrating in vivo persistence and tumor trafficking of mRNA MESO CAR T-cells.

Immunological activity was supported by demonstration of induction of humoral epitope spreading following mRNA MESO CAR T-cell infusions. This early clinical study of CAR-T therapy directed against MESO strongly supports the feasibility, safety and anti-tumor activity to progress with further development of this promising approach.

Integral to the development of mRNA CAR T-cell approaches has been the development of the MaxCyte GT™ highly effective system for ex vivo cell engineering. This system was utilized for the manufacture of the mRNA MESO CAR T-cells in the University of Pennsylvania clinical study. The MaxCyte GT™ system allows automated, robust, current Good Manufacturing Practice (cGMP) cell processing and manufacture in a closed system that can be completed in a few hours at any clinical facility that is equipped for hematopoietic cell processing. Using this system, any mRNA-modified CAR T-cell, known as CARMA, can be produced for potential clinical testing for antigen-specific CAR T-cell therapy. Similar to the prior work with Meso-targeted CAR T-cells, CARMA specific to human mesothelin (MCY-M11) provides a unique opportunity to develop a clinically effective and well-tolerated cell immunotherapy for patients with MESO-expressing malignancies, incorporating all of the safety, efficacy, and cell manufacturing advantages identified in preclinical and clinical studies.

### Pharmaceutical Compositions

Pharmaceutical compositions of transfected cells for administration to a subject are contemplated by the present disclosure. One of ordinary skill in the art would be familiar with techniques for administering cells to a subject. Furthermore, one of ordinary skill in the art would be familiar with techniques and pharmaceutical reagents necessary for preparation of these cell prior to administration to a subject. In certain embodiments of the present disclosure, the pharmaceutical composition will be an aqueous composition that includes the transfected cells that have been modified to transiently express the CAR. In certain embodiments, the transfected cell is prepared using cells that have been obtained from the subject (i.e., autologous cells). In certain embodiments, the transfected cell is prepared using cells that have been obtained from a donor (i.e., allogenic cells). In certain embodiments, the transfected cell is prepared using cells that have been obtained from a cell culture. Pharmaceutical compositions of the present disclosure comprise an effective amount of a solution of the transfected cells in a pharmaceutically acceptable carrier or aqueous medium. As used herein, “pharmaceutical preparation” or “pharmaceutical composition” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the transfected cancer cells, its use in the therapeutic compositions is contemplated. Supplementary active ingredients can also be incorporated into the compositions. For human administration, preparations should meet sterility, pyrogenicity, general safety and purity standards as required by the FDA Center for Biologics. The transfected cancer cells may be formulated for administration by any known route, such as by sub cutaneous injection, intramuscular injection, intravascular injection, intratumoral injection, intravenous injection, pleural administration, topical application, intraperitoneal injection, or application by any other route. A person of ordinary skill in the art would be familiar with techniques for generating sterile solutions for injection or application by any other route.

### Administration

The compositions of the present disclosure may be administered via any appropriate means. In some embodiments, the nucleic acid is administered transdermally, via injection, intramuscularly, subcutaneously, orally, nasally, intra-vaginally, rectally, transmucosally, enterally, parenterally, topically (e.g. at a post-surgical site), epidurally, intracerebrally intracerebroventricularly, intra-arterially, intra-articularly, intradermally, intralesionally, intraocularly, intraosseously, intraperitoneally, intrathecally, intrauterinely, intravenously, intravesical infusion, or intravitreally. Preferred routes of administration are intraperitoneally or intravenously.

In some embodiments, the route of administration depends on the disease being treated. In some embodiments, intravenous administration may be preferred for treatment of epithelial ovarian cancer, primary peritoneal cancer, fallopian tube carcinoma, peritoneal mesothelioma, pleural mesothelioma, non-small cell lung cancer (squamous or non-squamous), triple negative breast cancer, colorectal cancer, biliary tract cancer, gastric cancer, gastroesophageal cancer, pancreatic cancer, and thymic carcinoma. In other embodiments, intraperitoneal administration may be preferred for treatment of epithelial ovarian cancer, primary peritoneal cancer, fallopian tube carcinoma, and peritoneal mesothelioma.

Determination of the number of cells to be administered will be made by one of skill in the art, and will in part be dependent on the extent and severity of cancer, and whether the transfected cells are being administered for treatment of existing cancer or prevention of cancer. The preparation of the pharmaceutical composition containing the transfected cells will be known to those of skill in the art in light of the present disclosure.

Any number of cells in an appropriate dose may be administered. In some embodiments, transfected cells are administered at a dose of about 1×10⁷ to about 1×10¹⁰ cells. In some embodiments, transfected cells are administered at a dose of about 1×10⁷, about 5×10⁷ cells, about 1×10⁸, about 5×10⁸ cells, about 1×10⁹, about 5×10⁹ cells, about 1×10¹⁰ cells, or more per dose. In some embodiments, the dose is about 1×10⁷ cells. In some embodiments, the dose is about 5×10⁷ cells. In some embodiments, the dose is about 1×10⁸ cells. In some embodiments, the dose is about 5×10⁸ cells.

The transfected cells may be administered with other agents that are part of the therapeutic regimen of the subject, such as other immunotherapy, checkpoint inhibitors, immuno-oncology drugs, targeted agents, chemotherapy, and/or radiation. Examples of agents/therapeutic regimens that may be used in combination with the compositions of the present disclosure include, but are not limited to, drugs that block CTLA-4, PD-1, and/or PD-L1, CSF-1R inhibitors, TLR agonists, nivolumab, pembrolizumab, ipilimumab, atezolizumab, alemtuzumab, avelumab, ofatumumab, nivolumab, pembrolizumab, rituximab, durvalumab, cytokine therapy, interferons, interferon-α, interleukins, interleukin-2, dendritic cell therapy (e.g. Sipuleucel-T), CHOP, cyclophosphamide, methotrexate, 5-fluorouracil, vinorelbine, doxorubicin, docetaxel, bleomycin, dacarbazine, mustine, procarbazine, prednisolone, etoposide, cisplatin, epirubicin, folinic acid, and oxaliplatin. The compositions of the disclosure may be administered before the additional agent(s), concurrently with the additional agent(s), or after the additional agent(s).

The present disclosure provides methods of chronically administering the compositions to a patient. In some embodiments, the patient receives three or more separate doses. In some embodiments, the doses chronically administered to the patient are the same at each administration. In some embodiments, the doses chronically administered to the patient differ at one or more instances of administration. In some embodiments, the first dose is the highest, and subsequent doses are lower. In some embodiments, the subsequent lower doses are the same. In some embodiments, the subsequent lower doses differ. In some embodiments, the first dose is the lowest, and subsequent doses are higher. In some embodiments, the subsequent higher doses are the same. In some embodiments, the subsequent higher doses differ. In some embodiments, each dose differs.

In some embodiments, the multiple doses have an additive effect on the immune system. In some embodiments, the multiple doses have a synergistic effect on the immune system. Without being bound by theory, in some embodiments, the earlier doses break immune tolerance, and subsequent doses reactivate the immune system and then generate an immune cascade. In some embodiments, where three doses are administered, the first dose breaks immune tolerance, the second dose reactivates the immune system, and the third dose generates an immune cascade.

In certain aspects, the multiple doses may be chronically administered over a period of days, weeks, months, or year, or more. In some embodiments, the doses are administered daily, weekly, bimonthly, monthly, every other month, every 6 months, every year, or more. A subject may receive, for example, 1, 2, 3, 4, 5, 6, 7, 8, 9, 10, 11, 12, 13, 14, 15, 16, 17, 18, 19, or 20 or more doses. In some embodiments, the dose is administered weekly. In some embodiments, the dose is administered weekly for one to 52 weeks. In some embodiments, the dose is administered weekly for at least 2 weeks, at least 3 weeks, at least 4 weeks, at least 5 weeks, at least 6 weeks, at least 7 weeks, at least 8 weeks, at least 9 weeks, at least 10 weeks, or more. In some embodiments, a subsequent dose is not administered until after the cells of the previous dose no longer express a CAR. In some embodiments, a subsequent dose is not administered until one day, three days, or seven days after the previous dose. In some embodiments, the subsequent dose is administered while the previous dose still expresses a CAR. In some embodiments, a subsequent dose is administered less than one day, less than three days, or less than seven days after the previous dose is administered.

In some embodiments, the patient is administered about 1×10⁷ cells per dose weekly for three weeks (total dose amount of about 3×10⁷ cells). In some embodiments, the patient is administered about 51×10⁷ cells per dose weekly for three weeks (total dose amount of about 15×10⁷ cells). In some embodiments, the patient is administered about 1×10⁸ cells per dose weekly for three weeks (total dose amount of about 3×10⁸ cells). In some embodiments, the patient is administered about 5×10⁸ cells per dose weekly for three weeks (total dose amount of about 15×10⁸ cells).

The transfected cells of the disclosure may be chronically administered until the patient relapses or shows signs of disease progression, shows symptom improvement, tumor size or load decreases (e.g. partial response), cancer biomarker expression changes, the patient shows a complete response, or patient quality of life improves. These metrics may be measured by any appropriate means including, but not limited to, imaging (e.g. CAT scans, MRI), observation of lesions, biomarker assays (e.g. CA125 tests), or questionnaires.

The transfected cells may be administered to the subject at or near a tumor in the subject, or to a site from which a tumor has been surgically removed from the subject. In other embodiments, the transfected cells are administered locally to a tumor site, such as by intratumoral injection. However, it is not necessary that the transfected cells be administered at the tumor site to achieve a therapeutic effect. Thus, in certain embodiments the transfected cells may be administered at a site distant from the tumor site. A medical practitioner will be able to determine a suitable administration route for a particular subject based, in part, on the type and location of the hyperproliferative disease. The transfected cells may be administered locally to a disease site, regionally to a disease site, or systemically. In some embodiments, the cells are administered by intravenous injection, intraperitoneal injection, or intralymphatic injection.

In some embodiments, the transfected cells are administered to the patient within two weeks from the time the peripheral blood was collected (e.g. from the donor or from the same subject). In some embodiments, the transfected cells are administered to the patient between 2 weeks to about 1 hour from the time the peripheral blood was collected. In some embodiments, the transfected cells are administered back in to the patient in less than 48 hours, less than 24 hours, or less than 12 hours from the time from when the peripheral blood was collected. In certain aspects of the disclosure, the transfected cells are administered back in to the patient within about 1 to 48 hours, about 1 to 24 hours, about 1 to 15 hours, about 1 to 12 hours, about 1 to 10 hours, or about 1 to 5 hours from the time the peripheral blood is were collected The donor and the subject being treated may be the same person or different people. Thus, in some embodiments the cells are autologous to the subject; and in other embodiments, the cells are allogenic to the subject.

In some embodiments, administration of the transfected cells disclosed herein prevent, ameliorate, decrease, or delay tumor growth in a treated patient compared with controls or patients treated with other treatments, or the same patient before treatment. In some embodiments, tumor growth is prevented, ameliorated, decreased, or delayed in the treated patient between day 1 and year 10 compared with controls or patients treated with other treatments, or the same patient before treatment. In some embodiments, administration of the transfected cells disclosed herein prevents, ameliorates, decreases, or delays tumor growth at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with tumor growth in controls or patients treated with other treatments, or the same patient before treatment. In some embodiments, administration of the transfected cells disclosed herein prevents, ameliorates, decreases, or delays tumor growth for about 1 day, about 1 week, about 1 month, about 2 months, about 3 months, about 4 months, about 5 months, about 6 months, about 1 year, about 2 years, about 5 years, or about 10 years, or more compared with tumor growth in controls or patients treated with other treatments, or the same patient before treatment.

In some embodiments, tumor growth is decreased by about 1%, about 5%, about 10%, about 20%, about 30%, about 40%, about 50%, about 60%, about 70%, about 80%, about 90%, or about 100% compared with controls or patients treated with other cancer treatments, or the same patient before treatment. In some embodiments, administration of the transfected cells disclosed herein reduces tumor growth by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients treated with other cancer treatments, or the same patient before treatment. In some embodiments, administration of the transfected cells disclosed herein reduces tumor growth by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more compared with controls or patients treated with other cancer treatments, or the same patient before treatment.

In some embodiments, administration of transfected cells disclosed herein reduces cancer biomarker expression in a treated patient compared with controls or patients treated with other treatments, or the same patient before treatment. In some embodiments, administration of the transfected cells disclosed herein reduce cancer biomarker expression in a treated patient between day 1 and year 10 compared with controls or patients treated with other treatments, or the same patient before treatment. In some embodiments, administration of the transfected disclosed herein reduces cancer biomarker expression at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients treated with other cancer treatments, or the same patient before treatment. In some embodiments, administration of the transfected cells disclosed herein reduces cancer biomarker expression for about 1 day, 2 days, 3 days, 4 days, 5 days, 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years, or more compared with controls or patients treated with other cancer treatment, or the same patient before treatment.

In some embodiments, cancer biomarker expression is decreased by about 1%, about 5%, about 10%, about 20%, about 30%, about 40%, about 50%, about 60%, about 70%, about 80%, about 90%, or about 100% compared with controls or patients treated with other treatments, or the same patient before treatment. In some embodiments, administration of the transfected cells disclosed herein reduces cancer biomarker expression by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients treated with other cancer treatments, or the same patient before treatment. In some embodiments, administration of the transfected cells disclosed herein reduces cancer biomarker expression by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more compared with controls or patients treated with other cancer treatments or the same patient before treatment. In some embodiments, the cancer biomarker is a cytokine, a chemokine, a cell phenotype (e.g. as measured by FACS), mesothelin expression, Megakaryocyte Potentiating Factor (MPF), a tumor antigen, and/or a tumor associated antigen.

In some embodiments, administration of transfected cells disclosed herein reduces tumor size in a treated patient compared with controls or patients treated with other treatments, or the same patient before treatment. In some embodiments, administration of the transfected cells disclosed herein reduces tumor size in a treated patient between day 1 and year 10 compared with controls or patients treated with other treatments, or the same patient before treatment. In some embodiments, administration of the transfected disclosed herein reduces tumor size at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients treated with other cancer treatments, or the same patient before treatment. In some embodiments, administration of the transfected cells disclosed herein reduces tumor size for about 1 day, 2 days, 3 days, 4 days, 5 days, 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years, or more compared with controls or patients treated with other cancer treatment, or the same patient before treatment.

In some embodiments, tumor size is decreased by about 1%, about 5%, about 10%, about 20%, about 30%, about 40%, about 50%, about 60%, about 70%, about 80%, about 90%, or about 100% compared with controls or patients treated with other cancer treatments, or the same patient before treatment. In some embodiments, administration of the transfected cells disclosed herein reduces tumor size by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients treated with other cancer treatments, or the same patient before treatment. In some embodiments, administration of the transfected cells disclosed herein reduces tumor size by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more compared with controls or patients treated with other cancer treatments or the same patient before treatment.

In some embodiments, administration of transfected cells disclosed herein improves cancer symptoms in a treated patient compared with controls or patients treated with other treatments, or the same patient before treatment. In some embodiments, administration of the transfected cells disclosed herein improves cancer symptoms in a treated patient between day 1 and year 10 compared with controls or patients treated with other treatments, or the same patient before treatment. In some embodiments, administration of the transfected disclosed herein improves cancer symptoms at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients treated with other cancer treatments, or the same patient before treatment. In some embodiments, administration of the transfected cells disclosed herein improves cancer symptoms for about 1 day, 2 days, 3 days, 4 days, 5 days, 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years, or more compared with controls or patients treated with other cancer treatment, or the same patient before treatment.

In some embodiments, cancer symptoms are improved by about 1%, about 5%, about 10%, about 20%, about 30%, about 40%, about 50%, about 60%, about 70%, about 80%, about 90%, or about 100% compared with controls or patients treated with other cancer treatments, or the same patient before treatment. In some embodiments, administration of the transfected cells disclosed herein improves cancer symptoms by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients treated with other cancer treatments, or the same patient before treatment. In some embodiments, administration of the transfected cells disclosed herein improves cancer symptoms by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more compared with controls or patients treated with other cancer treatments or the same patient before treatment.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1—Administration of Cells Transiently Expressing an Anti-Mesothelin CAR Decrease Tumor Size and Increase Survival in Nude Mice

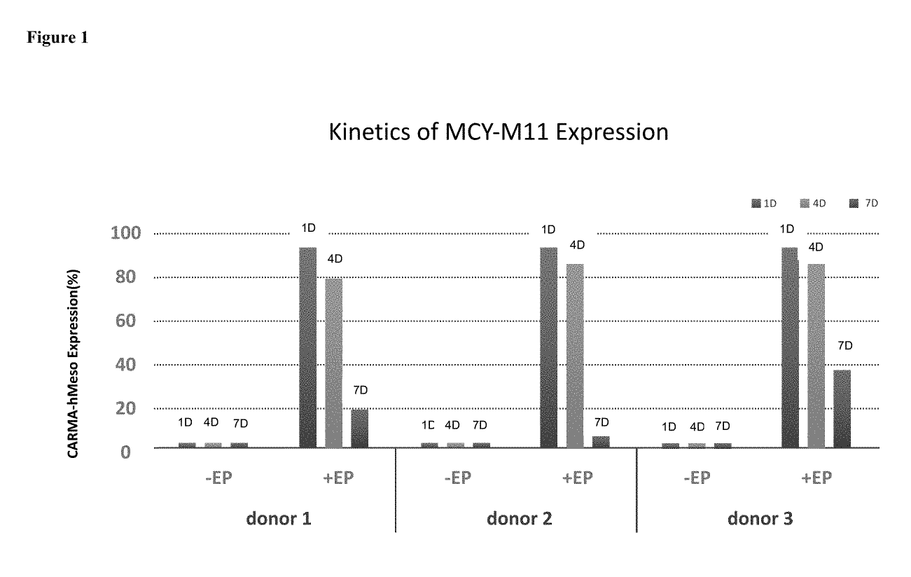

The present compositions (MCY-M11) demonstrate transient expression of an anti-mesothelin CAR in vitro, lasting approximately 7 days. Despite the short duration of expression, initial dosing is intended to break tolerance, re-activate the intact immune system, and generate an immune cascade. These activities are potentiated by subsequent (e.g. chronic) administration. FIG. 1 demonstrates the kinetics of MCY-M11 expression.

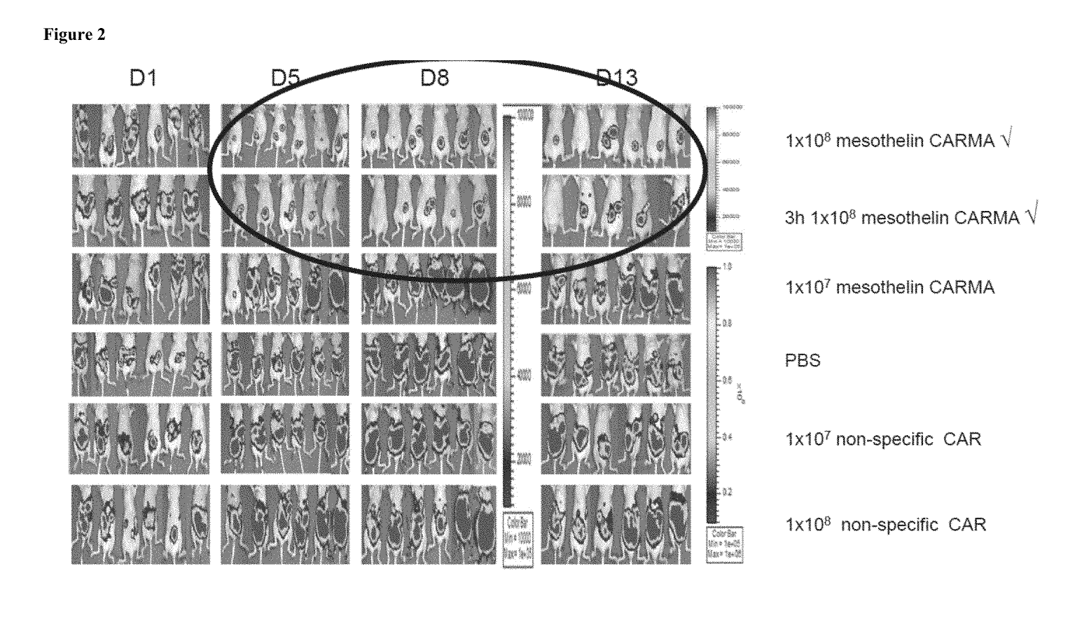

Groups of nude mice (N=6) were injected with ID8 ovarian tumor cells. One day after injection with the tumor cells, the animals were treated with: 1) 1×10⁸ mesothelin CARMA (i.e. MCY-M11); 2) 3 h 1×10⁸ mesothelin CARMA; 3) 1×10⁷ mesothelin CARMA; 4) PBS; 5) 1×10⁷ non-specific CAR; or 6) 1×10⁸ non-specific CAR. As shown in FIG. 2, administration of cells transiently expressing the anti-mesothelin CAR (MCY-M11) inhibits tumor growth.

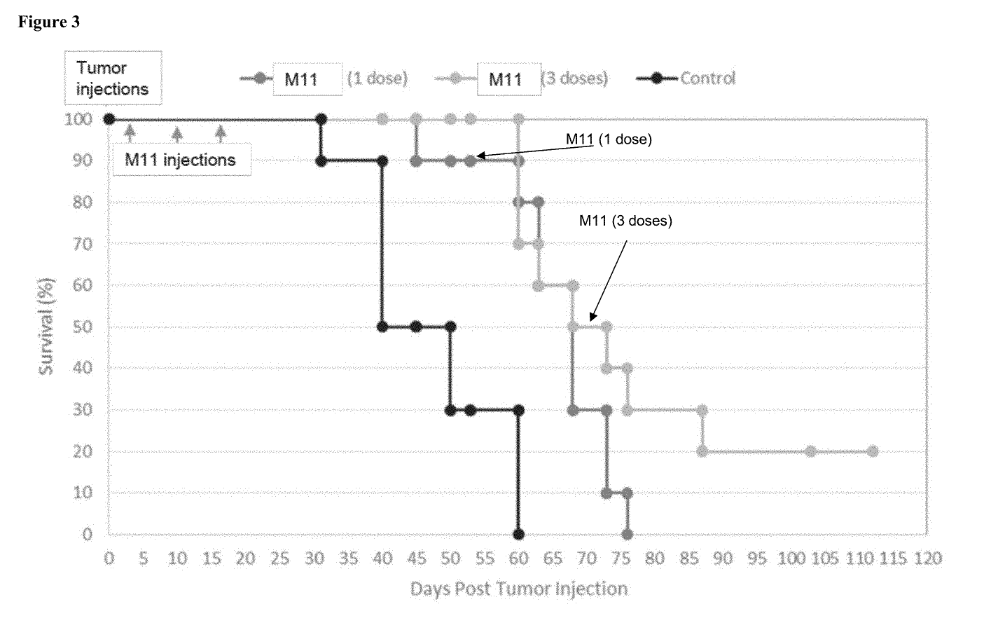

Further, as shown in FIG. 3, administration of cells transiently expressing the anti-mesothelin CAR (MCY-M11) increases survival of nude mice bearing solid tumors. Administration of one dose increases survival from 60 days to 75 days. Administration of three doses of the CAR cell further increases survival to over 110 days.

### Example 2—a Phase 1 Study of Intraperitoneal MCY-M11 Therapy for Women with Platinum Resistant High Grade Serous Adenocarcinoma of the Ovary, Primary Peritoneum, or Fallopian Tube, or Subjects with Peritoneal Mesothelioma with Recurrence after Priory Chemotherapy

Description of Drug:

MCY-M11 cells are non-expanded, autologous peripheral blood mononuclear cells (PBMCs) transfected with mRNA encoding the human CAR of contiguous peptide domains of scFV-αMeso-H, a transmembrane domain, 4-1BB, and CD3ζ signaling region (MCY-M11). MCY-M11 T-cells bind to mesothelin-expressing cells, with subsequent T-cell activation via CD3 and costimulatory molecule 4-1BB, to activate T-cell dependent antitumor activity. MCY-M11 offers the benefit of a greater safety profile compared to viral vector engineered CAR T therapies, as the cells have a limited lifespan. Additionally, the manufacture and timeline to therapeutic administration is more reliable and faster than CAR T-cells requiring viral vector engineering.

MCY-M11 cells were produced in the MaxCyte GT™ closed system using freshly isolated human PBLs that have been transfected with the CAR construct targeting human mesothelin. See U.S. Pat. No. 9,669,058 which is incorporated by reference in its entirety for all purposes. Using MaxCyte GT™, transfection of mRNA CAR in up to 20×10⁹ peripheral blood mononuclear cells (PBMCs) for clinical scale manufacture of CARMA has been reliably demonstrated. The cryopreserved product exhibited expression of MCT-M11 in >95% of cells, and was able to recognize and lyse tumor cells in an antigen-specific manner. Expression of MCY-M11 was detectable over approximately 7-10 days in vitro with a progressive decline of MCY-M11 expression that correlated with in vitro cell expansion.

This study was the first-in-human study of IP administration of MCY-M11 cells in human subjects.

In preclinical in vitro and in vivo studies of MCY-M11 cells using a preclinical model of ovarian cancer, MCY-M11 cells demonstrated high viability and CAR-expression, with the ability to recognize and kill mesothelin-expressing tumor cells at very low effector-to-target ratios. A single IP injection of MCY-M11 cells in a human ovarian cancer nude mouse model demonstrated a dose-dependent inhibition of tumor growth, with longer overall survival benefit compared to untreated control and CARMA-CD19 (irrelevant CAR) treated groups. Weekly administration of MCY-M11 cells over 1, 3, and 6 weeks extended disease control, resulting in increased overall survival compared with a single administration of MCY-M11 cells. Further, there were no overt toxicities noted in the human ovarian cancer nude mouse models following a single IP administration of up to 1×10⁸ MCY-M11 cells, a single IV administration of up to 4×10⁷ MCY-M11 cells, or following a total of six, once weekly, IP administrations of 5×10⁷ MCT-M11 cells.

Primary Objective:

to characterize the feasibility, safety, and tolerability of MCY-M11 when administered as an intraperitoneal (IP) infusion for three weekly infusions.

Secondary Objectives:

1) To assess anti-tumor activity in subjects administered MCY-M11 (e.g. Response Evaluation Criteria in Solid Tumors (RECIST), Immune-related Response Evaluation Criteria in solid Tumors (irRECIST), CA 125). 2) To assess correlative endpoints, including tumor expression of mesothelin, serum and ascites cytokine levels, serum and ascites levels of mesothelin and megakaryocyte potentiating factor (MPF), tumor associated antigens, and blood and ascites fluorescence-activated cell sorting (FACS) phenotyping.

Subject Inclusion Criteria:

Subjects must be at least 18 years old, and able to undergo peripheral blood leukapheresis for ex vivo isolation of circulating leukocytes. Subjects must have successful placement of an intraperitoneal catheter/port for intraperitoneal (IP) delivery.

Study Design:

Study Size:

Approximately 15-24 subjects are enrolled in this Phase 1 study to define a dose suitable for phase 2 testing by IP delivery. Several subjects have already been enrolled and dosing has started.

Dose Level Escalation Groups:

The dose escalation design follows a standard 3+3 approach. A cycle of MCY-M11 treatment consists of 3 weekly doses (i.e. three doses administered once a week). Subjects receive only 1 cycle of treatment of 3 infusions, regardless of treatment response.

Subjects were enrolled into 1 of 4 dose levels with a fixed dose level per group, and with dose escalation per group, based on a standard 3+3 dose escalation design:

Dose Level 1—1.0×10⁷ cells/dose for weekly dosing×3 doses

Dose Level 2—5.0×10⁷ cells/dose for weekly dosing×3 doses

Dose Level 3—1.0×10⁸ cells/dose for weekly dosing×3 doses

Dose Level 4—5.0×10⁸ cells/dose for weekly dosing×3 doses

Additional dose levels may be added during the course of the study. The decision for additional dose levels are based on the review of the totality of data from previous dose levels.

For Dose Levels 1, 2, and 3, a minimum of 3 subjects were enrolled.

The first 2 study subjects completed the entire cycle of treatment (3 weekly doses) plus 14 days before the next subject may begin dosing. After the second study subject has completed the entire cycle of treatment (3 weekly doses) plus 14 days, subsequent subjects may begin dosing no sooner than 14 days after the start of dosing for the previous subject.

MCY-M11 Administration

MCY-M11 was administered weekly for 3 weeks by IP delivery. An IP catheter/port was placed prior to start of treatment, with choice of catheter/port and care determined by the local site. Subjects who have complications of catheter/port placement were withdrawn from the study and replaced.

Results

Thus far, two patients have received three weekly doses of 1.0×10⁷ cells per dose (total dose amount was 3.0×10⁷ cells for each patient). No Grade 3 or Grade 4 toxicities or any SAEs have been observed.

### INCORPORATION BY REFERENCE

All publications, patents, and patent publications cited are incorporated by reference herein in their entirety for all purposes.

This application incorporates by reference the following publications and applications in their entireties for all purposes: U.S. Provisional Application No. 61/043,653 filed Apr. 9, 2008; U.S. Pat. No. 8,450,112 filed on Apr. 9, 2009; U.S. Pat. No. 9,132,153 filed on May 24, 2013; and U.S. Pat. No. 9,669,058, filed Aug. 25, 2015.

### REFERENCES

1. Lee, D. W., et al., Current concepts in the diagnosis and management of cytokine release syndrome. Blood, 2014. 124(2): p. 188-95.
2. Hassan, R., et al., Localization of mesothelin in epithelial ovarian cancer. Appl Immunohistochem Mol Morphol, 2005. 13(3): p. 243-7.
3. Gordon, A. N., et al., Phase II study of liposomal doxorubicin in platinum- and paclitaxel-refractory epithelial ovarian cancer. J Clin Oncol, 2000. 18(17): p. 3093-100.
4. Mutch, D. G., et al., Randomized phase III trial of gemcitabine compared with pegylated liposomal doxorubicin in patients with platinum-resistant ovarian cancer. J Clin Oncol, 2007. 25(19): p. 2811-8.
5. Markman, M., et al., Phase II trial of weekly single-agent paclitaxel in platinum/paclitaxel-refractory ovarian cancer. J Clin Oncol, 2002. 20(9): p. 2365-9.
6. Bridda, A., et al., Peritoneal mesothelioma: a review. MedGenMed, 2007. 9(2): p. 32.
7. Hassan, R., et al., Mesothelin Immunotherapy for Cancer: Ready for Prime Time? J Clin Oncol, 2016. 34(34): p. 4171-4179.
8. Morello, A., M. Sadelain, and P. S. Adusumilli, Mesothelin-Targeted CARs: Driving T Cells to Solid Tumors. Cancer Discov, 2016. 6(2): p. 133-46.
9. Zhao, X. Y., et al., Novel Antibody Therapeutics Targeting Mesothelin In Solid Tumors. Clin Cancer Drugs, 2016. 3(2): p. 76-86.
10. Beatty, G. L., et al., Mesothelin-specific chimeric antigen receptor mRNA-engineered T cells induce anti-tumor activity in solid malignancies. Cancer Immunol Res, 2014. 2(2): p. 112-20.
11. Beatty G L, O. H. M., Nelson AM, McGarvey M, Torigian D A, Lacey S F, Melenhorst J J, Levine B L, Plesa G, June C H. Safety and Antitumor Activity of Chimeric Antigen Receptor Modified T cells in Patients with Chemotherapy Refractory Metastatic Pancreatic Cancer. in ASCO Annual Meeting. 2015.
12. Zhao, Y., et al., Multiple injections of electroporated autologous T cells expressing a chimeric antigen receptor mediate regression of human disseminated tumor. Cancer Res, 2010. 70(22): p. 9053-61.
13. Brudno, J. N. and J. N. Kochenderfer, Toxicities of chimeric antigen receptor T cells: recognition and management. Blood, 2016. 127(26): p. 3321-30.
14. Eisenhauer E A, T. P., Bogaerts J, Schwartz L H, Sargent D, Ford R, et al., New response evaluation criteria in solid tumours: revised RECIST guideline (Version 1.1). Eur J Cancer, 2008(45): p. 228-47.
15. Wolchock J D, H. A., O'Day S, Weber J S, Hamid O, Lebbe C, Maio M, Binder M, Bohnsack O, Nichol G, Humphrey R, Hodi F S, Guidelines for the Evaluation of Immune Therapy Activity in Solid Tumors: Immune-related Response Criteria. 2009(15): p. 7412-7420.
16. Folstein, M. F., S. E. Folstein, and P. R. McHugh, “Mini-mental state”. A practical method for grading the cognitive state of patients for the clinician. J Psychiatr Res, 1975. 12(3): p. 189-98.
17. Oken, M. M., et al., Toxicity and response criteria of the Eastern Cooperative Oncology Group. Am J Clin Oncol, 1982. 5(6): p. 649-55.
18. Landi A, Babiuk L A, van Drunen Littel-van den Hurk S. High transfection efficiency, gene expression, and viability of monocyte-derived human dendritic cells after nonviral gene transfer. J Leukoc Biol. 2007.
19. Van De Pane T J, Martinet W, Schrijvers D M, Herman A G, De Meyer G R. mRNA but not plasmid DNA is efficiently transfected in murine J774A.1 macrophages. Biochem Biophys Res Commun. 2005; 327:356-360.
20. Rabinovich P M, Komarovskaya M E, Ye Z J, et al. Synthetic messenger RNA as a tool for gene therapy. Hum Gene Ther. 2005; 17:1027-1035.
21. Zhao Y, Zheng Z, Cohen C J, et al. High-efficiency transfection of primary human and mouse T lymphocytes using RNA electroporation. Mol Ther. 2006; 13:151-159.
22. Schwartz, Annu. Rev. Immunol., 21:305-334, 2003.
23. Ramsdell et al., Science, 246:1038-1041, 1989.
24. Staveley-O'Carroll, et al., Proc. Natl. Acad. Sci. USA, 95:1178-83, 1998.
25. Fearon E R, Vogelstein B: A genetic model for colorectal tumorigenesis. Cell 1; 61(5):759-767, 1990
26. Califano et al. Genetic Progression Model for Head and Neck Cancer: Implications for Field Cancerization. Cancer Res Jun. 1 1996 (56) (11) 2488-2492.

1. A method of treating cancer by chronically administering more than one dose of a population of modified unstimulated mononuclear cells, wherein the unstimulated mononuclear cells are obtained from peripheral blood and transfected with an mRNA encoding a chimeric antigen receptor.

2. The method of claim 1, wherein the dose is repeated daily, weekly, or monthly.

3. The method of claim 2, wherein the dose is repeated weekly.

4. The method of claim 3, wherein the dose is repeated weekly for three weeks.

5. The method of claim 1, wherein the dose is 1×10⁷ or 5×10⁷ cells.

6. The method of claim 1, wherein the chimeric antigen receptor comprises an antigen-binding region, a 4-1BB costimulatory signaling region, and a CD3zeta signaling region.

7. The method of claim 6, wherein the antigen-binding region is an scFv.

8. The method of claim 1, wherein the antigen-binding region binds to a tumor antigen.

9. The method of claim 1, wherein the cancer is selected from the group consisting of breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, leukemia, acute lymphocytic leukemia (ALL), acute myelogenous leukemia (AML), chronic lymphocytic leukemia (CLL), chronic myelogenous leukemia (CML), and mantle cell lymphoma (MCL).

10. The method of claim 1, wherein the tumor antigen is selected from the group consisting of CD-19, FBP, TAG-72, CEA, CD171, IL-13 receptor, G(D)2, PSMA, mesothelin, Lewis-Y, and CD30.

11. The method of claim 6, wherein the chimeric antigen receptor comprises an anti-mesothelin binding-region.

12. The method of claim 11, wherein the anti-mesothelin binding region is an scFv.

13. The method of claim 1, wherein the mononuclear cells are selected from the group consisting of B cells, T cells, Natural Killer cells, or PBMCs.
